(19) United States
(12) Patent Application Publication
(10) Pub. No.: US 2006/0127336 A1
(43) Pub. Date: Jun. 15, 2006

(54) Deodorants

(75) Inventors: Kazuyuki Harima, Wakayama-shi (JP); Takanori Kotera, Wakayama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KAO CORPORATION, Tokyo (JP)

(21) Appl. No.: 11/009,133

(22) Filed: Dec. 13, 2004

(51) Int. Cl. A61K 8/26

(52) U.S. Cl. 424/68

(57) ABSTRACT

A method of deodorization comprising allowing an odor-causing substance to coexist an aluminosilicate particle having the composition of a M₂O.Al₂O₃.b SiO₂.c RₘQₙ.d H₂O, wherein M is one or more members selected from the group consisting of Na, K and H, R is one or more members selected from the group consisting of Na, K, Ca and Mg, Q is one or more members selected from the group consisting of CO₃, SO₄, NO₃, OH and Cl, a satisfies 0<a≦1, b satisfies 1≦b≦50, c satisfies 0<c≦2, d satisfies d≧0, m satisfies 1≦m≦2, and n satisfies 1≦n≦2, and a specific surface area of 70 to 800 m²/g. There is provided a deodorization using aluminosilicate particles, which have a wide deodorizing spectrum, are capable of effectively deodorizing an odor from various causative substances generated in daily life environment, are also safe to a human body, and furthermore exhibit excellent appearance upon application.

### FIELD OF THE INVENTION

The present invention relates to a deodorization using a specified aluminosilicate particle.

### BACKGROUND OF THE INVENTION

With the improvement of the living environment of recent years, there is an increasing desire of the removal of odor. Such odor includes, for instance, an alkaline odor from ammonia, amine or the like, an acidic odor from a lower fatty acid, a sulfur-containing compound odor from a mercaptan or the like, and a neutral odor from an ester, a ketone, or an aldehyde or the like. It is important to remove a wide variety of odors having different properties. As methods of removing odor, there have been known a masking method, an ozone oxidation method, a drug neutralization method, a microbial degradation method, an adsorption method and the like.

### SUMMARY OF THE INVENTION

The present invention provides a method of deodorization comprising allowing an odor-causing substance to coexist with an aluminosilicate particle having the composition of:
aM₂O.Al₂O₃.bSiO₂.cRₘQₙ.dH₂O,
wherein M is one or more members selected from the group consisting of Na, K and H, R is one or more members selected from the group consisting of Na, K, Ca and Mg, Q is one or more members selected from the group consisting of CO₃, SO₄, NO₃, OH and Cl, a satisfies 0<a≦1, b satisfies 1≦b≦50, c satisfies 0<c≦2, d satisfies d≧0, m satisfies 1≦m≦2, and n satisfies 1≦n≦2, and a specific surface area of 70 to 800 m²/g.

### DETAILED DESCRIPTION OF THE INVENTION

However, each of the above-mentioned methods has some disadvantages. For instance, the masking method cannot be said as a method of essentially removing an odor; ozone is used in the ozone oxidation method, thereby necessitating that facilities be large-scaled; in the drug neutralization method, a substance to be treated is limited to a neutralizable substance, thereby making an odor to be treated by the method limited; and the microbial degradation method does not give an immediate effect.

On the other hand, the adsorption method is a convenient method of deodorization having an immediate effect and being highly safe. An activated carbon is widely used as an adsorbent. However, the method has some disadvantages that the activated carbon has low deodorizing ability against ammonia, and that hygiene might be lowered when applied to a human body because of its black color. White deodorants include zeolite and activated clay, but their deodorizing ability is lower than that of the activated carbon.

The present invention relates to a method of deodorization comprising allowing an odor-causing substance to coexist with a pale colored, preferably white aluminosilicate particle which has a wide deodorization spectrum, and is capable of deodorizing odor from various causative substances generated in daily life environment, and also safe to a human body, and furthermore exhibits excellent appearance upon application.

These and other advantages of the present invention will be apparent from the following description.

The aluminosilicate particle in the present invention is not particularly limited in its embodiment upon use as long as the aluminosilicate particle is used in deodorization. The aluminosilicate particle may be described herein as a deodorant in some cases from the viewpoint of its use as a deodorizing component.

One of the great features of the aluminosilicate particle of the present invention resides in that the aluminosilicate particle has a specified composition and properties as described below. Since the aluminosilicate particle has such a constitution, the aluminosilicate particle is capable of exhibiting a wide deodorization spectrum, thereby having excellent deodorizing effects against various odors. In addition, since the aluminosilicate particle is a pale colored, preferably white aluminosilicate particle, the aluminosilicate particle is suitably used in sanitary use, and exhibits excellent appearance upon application.

Specifically, the deodorant of the present invention is composed of an aluminosilicate particle having the following composition:
aM₂O.Al₂O₃.bSiO₂.cRₘQₙ.dH₂O,
wherein M is one or more members selected from the group consisting of Na, K and H, R is one or more members selected from the group consisting of Na, K, Ca and Mg, Q is one or more members selected from the group consisting of CO₃, SO₄, NO₃, OH and Cl, a satisfies 0<a≦1, b satisfies 1≦b≦50, c satisfies 0<c≦2, d satisfies d≧0, m satisfies 1≦m≦2, and n satisfies 1≦n≦2. Since the deodorant as referred to herein is substantially composed of the aluminosilicate particle itself, various properties of the particle, which is a constituent of the deodorant, directly show the properties of the deodorant.

In the above formula, M is preferably Na, H or a mixture thereof, from the viewpoint of exhibition of a high deodorizing ability and economic advantage. When M is Na and H, aM₂O is represented by a₁Na₂O.a₂H₂O, with a proviso that a₁+a₂=a. In addition, R is preferably Na, from the same viewpoint as that of M. Q is preferably CO₃ or NO₃, from the viewpoint of facilitation in the control of particle shape.

Furthermore, a satisfies preferably 0<a≦0.5, more preferably 0<a≦0.25, from the viewpoint of improvement in ability of deodorizing an alkaline odorous gas. b satisfies preferably 1≦b≦40, more preferably 1≦b≦30, from the viewpoint of improvement in ability of deodorizing an acidic odorous gas. c satisfies preferably 0<c≦1, more preferably 0<c≦0.6, even more preferably 0<c≦0.3, from the viewpoint of exhibiting high deodorizing ability. d is a content (molar ratio) of water contained in the aluminosilicate particle, and varies depending on the existing forms of the aluminosilicate particle, for instance, powdery and slurry-like forms. m and n are arbitrarily determined by the combination of R and Q.

In addition, the aluminosilicate particle has a specific surface area of from 70 to 800 m²/g, preferably from 80 to 600 m²/g, more preferably from 100 to 500 m²/g, from the viewpoint of giving a suitable deodorizing rate and a wide deodorization spectrum. The specific surface area can be obtained according to the method described in Examples set forth below.

The feature of the aluminosilicate particle of the present invention resides in that the aluminosilicate particle has a large specific surface area as described above, and a large number of acid points. Such a feature is not found in the aluminosilicate particle used as the raw material of the deodorant of the present invention described below (hereinafter referred to as raw material aluminosilicate particle). For this reason, excellent effects are exhibited that a deodorization spectrum is wider, and a higher deodorizing ability is exhibited, as compared to the raw material aluminosilicate particle. This is presumably due to increase in specific surface area, increase in microporous capacity and increase in acid points accompanying acid treatments in the preparation of the aluminosilicate particle from the raw material aluminosilicate particle.

Also, the aluminosilicate particle has an acid content of preferably 20 meq/100 g or more, more preferably 100 meq/100 g or more, even more preferably 170 meq/100 g or more, from the viewpoint of improvement in ability of deodorizing an alkaline odorous gas.

The term “acid content” as referred to herein is a total content of acid points of the aluminosilicate particle composing the deodorant of the present invention. The acid content can be obtained according to the method described in the Examples set forth below.

In addition, the aluminosilicate particle composing the deodorant of the present invention has an average particle diameter of preferably from 1 to 500 μm, more preferably from 1 to 300 μm, even more preferably from 1 to 100 μm, within which range it is preferable from the viewpoint of deodorization rate and feel of use upon application to a human body. The average particle size is determined, for instance, with a laser/scattering-type particle size distribution analyzer (LA-920, commercially available from HORIBA, Ltd.) at a refractive index of 1.16.

Furthermore, the shape of the aluminosilicate particle is not particularly limited, and the shape is preferably an acicular, platy or columnar form, from the viewpoint of improvement in feel of use when applied to a human body and improvement in yield upon addition to a carrier, for instance, addition to paper, a nonwoven fabric or the like. The aluminosilicate particle may be amorphous or crystalline, without any particular limitation. The aluminosilicate particle is obtained as an aggregate of acicular crystals, platy crystals, or columnar crystals depending upon the preparation conditions. Alternatively, those crystals may be aggregated to form, for instance, a spherical, tetrapod-like or massive aggregate, or a secondary aggregate thereof.

The term “acicular form” as referred to herein is one having a thickness of 500 nm or smaller, and a length as defined by its aspect ratio relative to the thickness of 2.0 or larger, the term “platy form” is one having a thickness of 300 nm or smaller, and a platy diameter as defined by its aspect ratio relative to the thickness of 2.0 or larger, and the term “columnar form” is one having a thickness of 50 nm or larger, and a length as defined by its aspect ratio relative to the thickness of 1.0 or larger and smaller than 2.0.

It is desired that the aluminosilicate particle used in the present invention is obtained, for instance, by subjecting a raw material aluminosilicate particle having the composition of:
wM₂O.Al₂O₃.xSiO₂.yRₘQₙ.zH₂O,
wherein M is Na, K or a mixture thereof, R is one or more members selected from the group consisting of Na, K, Ca and Mg, Q is one or more members selected from the group consisting of CO₃, SO₄, NO₃, OH and Cl, w satisfies 0.1≦w≦3, x satisfies 0.2≦x≦6, y satisfies 0<y≦2, z satisfies z≧0, m satisfies 1≦m≦2, n satisfies 1≦n≦2
to an acid treatment.

In the above formula, M is preferably Na. When M is Na and K, wM₂O is expressed by w₁Na₂O.w₂K₂O, with the proviso that w₁+w₂=w. Also, R is preferably Na. Q is preferably CO₃ or NO₃. Further, w satisfies preferably 0.1≦w≦2.5, more preferably 0.2≦w≦2. x satisfies preferably 0.2≦x≦5, more preferably 0.5≦x≦4. y satisfies preferably 0<y≦1.8, more preferably 0.2<y≦1.5. z is the content (molar ratio) of water contained in the crystal of the raw material aluminosilicate particle. The raw material aluminosilicate particle has a specific surface area of smaller than 70 m²/g, preferably 65 m²/g or smaller from the viewpoint of facilitating the control of the particle shape and securing a high yield during the preparation. In addition, it is preferable that the raw material aluminosilicate particle has an average particle size of the same size as that of the aluminosilicate particle composing the deodorant of the present invention.

Furthermore, it is preferable that the raw material aluminosilicate particle has one or more of those having cancrinite-like X-ray diffraction patterns selected from the group consisting of Nos. 20-379, 20-743, 25-776, 25-1499, 25-1500, 30-1170, 31-1272, 34-176, 35-479, 35-653, 38-513, 38-514, 38-515 and 45-1373 in an X-ray powder diffraction file published by JCPDS (Joint Committee on Powder Diffraction Standards), from the viewpoint of facilitating the control of the particle form of the aluminosilicate particle as a deodorant obtained therefrom, exhibiting high deodorizing ability, especially exhibiting high deodorizing ability against an acidic odor and a neutral odor.

The process for preparing a raw material aluminosilicate particle used in the present invention is not particularly limited. The process for preparing a raw material aluminosilicate particle includes, for instance, a process including the step of reacting an alumina raw material and a silica raw material in an aqueous alkali solution in the presence of CO₃²⁻, SO₄²⁻, NO₃⁻, Cl⁻ or the like.

The alumina raw material includes, for instance, aluminum oxide, aluminum hydroxide, sodium aluminate and the like, and sodium aluminate is preferable. The silica raw material includes, for instance, silica sand, quartz rock, water glass, sodium silicate, silica sol and the like, and water glass is preferable. Alternatively, as a raw material used as both of the alumina raw material and the silica raw material, there may be used, for instance, a clay mineral such as kaolin, montmorillonite, bentonite, mica or talc, and an aluminosilicate mineral such as mullite.

The raw material of CO₃²⁻ includes, for instance, carbon dioxide gas, sodium carbonate, potassium carbonate, potassium sodium carbonate, calcium carbonate, magnesium carbonate and the like, and sodium carbonate is preferable. The raw material of SO₄²⁻ includes, for instance, sulfuric acid, sodium sulfate, potassium sulfate, potassium sodium sulfate and the like, and sulfuric acid and sodium sulfate are preferable. The raw material of NO₃⁻ includes, for instance, nitric acid, sodium nitrate, potassium nitrate and the like, and nitric acid and sodium nitrate are preferable. The raw material of Cl⁻ includes, for instance, hydrochloric acid, sodium chloride, potassium chloride and the like, and hydrochloric acid and sodium chloride are preferable.

As an alkali for the aqueous alkali solution, there can be used, for instance, an oxide such as sodium oxide or potassium oxide; a hydroxide such as sodium hydroxide or potassium hydroxide; a carbonate such as sodium carbonate, potassium carbonate or potassium sodium carbonate; a hydrogencarbonate such as sodium hydrogencarbonate or potassium hydrogencarbonate; or the like. There may be used as desired an oxide such as calcium oxide or magnesium oxide; a hydroxide such as calcium hydroxide or magnesium hydroxide; a carbonate such as calcium carbonate, magnesium carbonate or dolomite; a hydrogencarbonate such as calcium hydrogencarbonate or magnesium hydrogencarbonate; or the like.

The raw material aluminosilicate particle used in the present invention can be obtained by blending, mixing and reacting various compounds mentioned above in a given ratio. The blending ratio is appropriately determined depending on the composition of the resulting desired raw material aluminosilicate particle. Preferably, it is desired that the molar ratio of blending components as a raw material of a raw material aluminosilicate particle is such that M₂O/SiO₂ is preferably from 0.01 to 100, more preferably from 0.05 to 80, that Al₂O₃/SiO₂ is preferably from 0.01 to 10, more preferably from 0.05 to 8, that RₘQₙ/SiO₂ is preferably from 0.01 to 100, more preferably from 0.05 to 80, and that H₂O/M₂O is preferably from 0.01 to 100, more preferably from 0.05 to 80, when expressing the components as M₂O, Al₂O₃, SiO₂ and RₘQₙ on the basis of the constituting elements of each component.

Also, the reaction temperature in the preparation of the raw material aluminosilicate particle is preferably from 15° to 300° C., more preferably from 60° to 150° C., even more preferably from 80° to 130° C., from the viewpoint of increasing crystallinity of the raw material aluminosilicate particle and stabilizing its shape, and from the viewpoint of reducing chemical corrosion and pressure load on a reaction vessel. The reaction time is preferably 2 hours or longer and 48 hours or shorter, from the viewpoint of completely carrying out the crystallization reaction.

The solid content of the raw material aluminosilicate particle thus obtained is preferably from 0.1 to 50% by weight.

The aluminosilicate particle composing the deodorant of the present invention is obtained by subjecting the raw material aluminosilicate particle to an acid treatment, and the acid treatment refers to activation to make all or a part of the particle amorphous not only by eluting M₂O and RₘQₙ existing in the pore of the raw material aluminosilicate particle, but also by eluting a part of skeleton-forming Al, which has technical effects of attaining an increase in specific surface area, an increase in microporous capacity and an increase in acid points. Therefore, the deodorizing ability is remarkably improved in the aluminosilicate particle of the present invention as compared to that of the raw material aluminosilicate particle. An extent of the acid treatment of the raw material aluminosilicate particle may be appropriately adjusted so that the resulting aluminosilicate particle has a desired property.

In the acid treatment of the raw material aluminosilicate particle, it is preferable to use a strong acid such as a hydrochloric acid, sulfuric acid or nitric acid, especially preferably a hydrochloric acid or nitric acid.

The acid treatment is specifically carried out by adding an aqueous solution containing the above-mentioned acid to the raw material aluminosilicate particle gradually or at once, thereby contacting the particle with the acid. The acid may be added at a rate of preferably from 0.01 to 100 mL/min, more preferably from 0.1 to 10 mL/min per 100 g of the raw material aluminosilicate particle.

In the acid treatment, the raw material aluminosilicate particle is made into a slurry state. The solid content of the mixture is preferably from 1 to 50% by weight, from the viewpoint of securing flowability of the reaction mixture (slurry), and preventing imbalance in the acid treatment, thereby improving the treatment efficiency.

The temperature for the acid treatment is preferably from 60° to 150° C., more preferably 80° to 120° C., from the viewpoint of increase in specific surface area and reduction in chemical or pressure load on the reaction vessel. Also, it is preferable that the acid treatment is carried out while properly stirring. The time period for the acid treatment after contacting the raw material aluminosilicate with the acid is preferably from 0.01 to 100 hours, more preferably from 0.1 to 10 hours.

The mixing ratio of the raw material aluminosilicate particle and the acid upon the acid treatment is such that the acid is preferably from 0.3 to 3 molar equivalent, more preferably from 0.5 to 2.5 molar equivalent, even more preferably from 0.9 to 2.1 molar equivalent based on 100 g of the aluminosilicate particle, within which range is preferable since the raw material aluminosilicate particle becomes excellently amorphous and aluminum in the particle is not excessively eluted into water, reduction in increased acid points is not found, so that deodorizing ability of the deodorant of the present invention composed of the aluminosilicate particle obtained after the acid treatment is not lowered.

After the acid treatment, it is preferable that the reaction mixture is properly aged, for instance, at 60° to 150° C. for about 0.1 to 10 hours. Next, the slurry is filtered, and washed with water to remove excess ionic components. The filter used in the filtration is not particularly limited, and there can be used, for instance, a filter such as a Nutsche filter or filter press filter.

After washing with water, the resulting aluminosilicate particle can be immediately used as the deodorant of the present invention. Alternatively, the aluminosilicate particle may be subjected to a desired treatment depending on the embodiment upon use of the deodorant. The embodiment upon use includes a filtration cake, a slurry, a dry powder and the like. The embodiment upon use may be selected in consideration of the application of the deodorant, and conditions in blending the deodorant with other components to be added as desired. For instance, when the aluminosilicate particle is prepared into a dry powder, the aluminosilicate particle obtained may be dried appropriately with a drier. The drier which can be used herein is not particularly limited, and includes, for instance, a blast drier, a vacuum drier, a spray-drier and the like.

Incidentally, since there are some cases where a part of M is substituted with H in the composition as a consequence of the acid treatment of the raw material aluminosilicate particle, there are some cases where M is H in the aluminosilicate particle composing the deodorant of the present invention, while there are no cases where M is H in the raw material aluminosilicate particle. In addition, the compositional ratio (molar ratio) of each component of the raw material aluminosilicate particle is slightly changed by the acid treatment.

The deodorant of the present invention can be obtained by the process as described above. It is preferable that the aluminosilicate particle composing the deodorant further carries a metal such as Ag, Cu, Zn, Fe or Ce, especially one or more kinds of those specifically exemplified metals, from the viewpoint of giving antibacterial property, and further improving the ability of deodorizing a sulfur-based odor from a mercaptan, hydrogen sulfide or the like. The carrying amount of those metals in the deodorant composed of the aluminosilicate particle after the carrying of the metal is preferably from 0.1 to 30% by weight, more preferably from 0.1 to 10% by weight, from the viewpoint of exhibition of the desired effects and economic advantage. The carrying amount can be measured by a fluorescent X-ray determination method. The term “carrying” as used herein and any grammatical variation thereof mean binding of the above-mentioned metal element to the aluminosilicate particle by a physical and/or chemical binding force.

The method of allowing the aluminosilicate particle to carry a metal includes, for instance, a method including the step of subjecting the raw material aluminosilicate particle to an acid treatment in the presence of the metal-containing compound to allow the resulting aluminosilicate particle to carry a metal by ion exchange; a method including the steps of suspending a powder of the prepared deodorant in water, and adding an aqueous solution of a metal-containing compound thereto to allow the resulting aluminosilicate particle to carry a metal by ion exchange; and the like. Besides them, other methods of allowing the aluminosilicate particle to carry a metal include a general metal carrying method such as an immersion method or precipitation method.

The above-mentioned metal-containing compound is not particularly limited, as long as the compound is a water-soluble metal-containing compound containing a desired metal. The compound includes, for instance, a nitrate, a sulfate, and a chloride, each containing a desired metal.

In the case where the aluminosilicate particle carries a metal, a part of M is substituted with a metal in a composition of an aluminosilicate particle composing the deodorant of the present invention. Therefore, when a metal is represented by D, aM₂O is represented by a₁′ D₂O.a₂′M₂O, with the proviso that a₁′+a₂′=a, in the composition of the aluminosilicate particle after the metal carrying.

The deodorant of the present invention has a wide deodorization spectrum, and exhibits excellent deodorizing effect against, for instance, an alkaline odor from ammonia, amine, pyridine or the like, an acidic odor from a lower fatty acid or the like, a sulfur-containing compound odor from a mercaptan or the like, and further a neutral odor from an ester, a ketone, an aldehyde or the like. Among them, particularly excellent deodorizing ability can be exhibited against a sulfur-containing compound odor from methyl mercaptan, ethyl mercaptan, methyl sulfide, methyl disulfide, hydrogen sulfide or the like. In addition, highly excellent deodorizing ability can be exhibited on 3-mercapto-3-methylhexan-1-ol and the like which are causative substances of armpit odor.

In the method of deodorization using the aluminosilicate particle of the present invention, the deodorization is carried out by allowing the aluminosilicate particle to coexist with an odor-causing substance. Embodiments for carrying out deodorization using the aluminosilicate particle include an embodiment of allowing the aluminosilicate particle to exist in a room or in a space of a refrigerator having an odor; and an embodiment of adhering an aluminosilicate particle to a filter of a vacuum cleaner, an air conditioner, an air cleaner or the like. Additional embodiments include, for instance, an embodiment of allowing the aluminosilicate particle to exist in a fiber, thereby removing an odor adsorbed to clothes; an embodiment of allowing the aluminosilicate particle to exist in a fiber of paper or a nonwoven fiber of sanitary napkins, diapers and the like. The ratio of the aluminosilicate particle to a coexisting odor-causing substance is not particularly limited, and the ratio may be properly adjusted so as to give a desired extent of deodorization.

The deodorant of the present invention can be used in any given granular form or in the form of a molded article, such as a powdery, granular or pelletal form depending upon the desired use. When the deodorant is powdery, the deodorant does not have a roughened texture but has excellent feel of use when applied to a human body. On the other hand, when the deodorant has a granular or pelletal form, scattering of the deodorant can be suppressed, thereby having excellent handleability. In the molding of the deodorant into a granular form or molded article, there can be used an inorganic binder such as various clays or water glass, and an organic binder such as carboxymethyl cellulose, polyvinyl alcohol, various oils and various waxes. Furthermore, the deodorant of the present invention may be used as a mixture with an adsorbent, or a photocatalyst, wherein the adsorbent includes an activated clay, an activated carbon, silica gel, hydrotalcite, a clay mineral or titanium oxide. Therefore, as one embodiment of the present invention, the deodorant of the present invention may be used as a deodorizing composition containing the deodorant and other components mentioned above which are added depending upon use. The content of the deodorant of the present invention in the deodorizing composition is preferably from 1 to 50% by weight. This composition has an excellent deodorizing ability of the same level as that of the deodorant of the present invention.

One preferred example of the embodiment upon use of the deodorant of the present invention includes a body deodorant. The form of the body deodorant includes pump spray, stick, gel, soft solid, roll-on, powder spray, cream, lotion, powder and sheet, and can be designed without any particular limitation. In these uses, the body deodorant can be prepared by properly blending the deodorant of the present invention together with known components which are used in the uses. The content of the deodorant of the present invention in each of those body deodorants is preferably from 0.01 to 50% by weight, more preferably from 0.1 to 30% by weight, even more preferably from 0.3 to 10% by weight.

### EXAMPLES

The following examples further describe and demonstrate embodiments of the present invention. The examples are given solely for the purposes of illustration and are not to be construed as limitations of the present invention.

The methods for determination of properties of the samples used in Examples and Comparative Examples are summarized hereinbelow.

(Method for Determination of Specific Surface Area)

The specific surface area was determined with FlowSorb Model 2300 (commercially available from Shimadzu Corporation). The sample used was 0.1 g, and a mixed gas of N₂/He=30/70 (volume ratio) was used as an adsorbing gas.

(Method for Determination of Acid Content)

A 0.5 g sample was added to 100 mL of a 0.01 mol/L aqueous NaOH, and the mixture was stirred for 1 hour. Thereafter, the resulting sample suspension was centrifuged (10000 rpm for 5 minutes), and 25 mL of supernatant was collected. This supernatant was titrated with 0.01 mol/L HNO₃ to obtain an amount of NaOH consumed, and an acid content of the sample was calculated based on the value obtained.

(Method for Determination of Deodorizing Ability)

A 0.1 g sample was sealed into a 3 L Tedlar bag (manufactured by Sansho Co., Ltd.), and the bag was filled with 3 L of an odorous gas of which concentration was adjusted at room temperature (25° C.). After a given time period passed, the concentration of the odorous gas in the bag was determined with a gas detecting tube (commercially available from Gastec Corporation). The odorous gas component and the time period from filling the odorous gas to determining the concentration of the odorous gas (determination time) are shown in Tables 1 to 4, respectively. Here, 0 minute is an initial concentration.

### Example 1

To a solution prepared by dissolving 103 g of sodium hydroxide in 1000 mL of ion-exchanged water, and further mixing therewith 157 g of a sodium aluminate solution (Na₂O=19.8% by weight, Al₂O₃=25.9% by weight, H₂O=54.3% by weight) was added 259 g of water glass (Na₂O=9.8% by weight, SiO₂=29.6% by weight, H₂O=60.6% by weight) over 1 minute, and the components were reacted at 100° C. for 2 hours. Thereafter, a solution obtained by mixing a solution prepared by dissolving 32 g of sodium hydroxide in 110 mL of ion-exchanged water, with 124 g of nitric acid (61%) was additionally added thereto over 1 minute, and the components were further reacted at 100° C. for 10 hours. After the reaction, the formed aluminosilicate particles were filtered and washed, and dried at 105° C. for 12 hours to give a powder of raw material aluminosilicate particles. The resulting raw material aluminosilicate particles were aggregates of columnar and acicular crystals to have a grown form into a tetrapod-like shape. The resulting raw material aluminosilicate particles were subjected to X-ray diffraction using an X-ray powder diffractometer [RINT2500, commercially available from Rigaku Corporation]. As a result, the aluminosilicate particles had diffraction patterns corresponding to JCPDS No. 38-513. The composition of the aluminosilicate particles was Na₂O.Al₂O₃.2.3 SiO₂.0.7 NaNO₃.1.3 H₂O, and their specific surface area was 10 m²/g.

One-hundred grams of the resulting raw material aluminosilicate particles were suspended in 900 mL of ion-exchanged water, and the resulting mixture was kept at 100° C. While stirring, 120 mL of 61% nitric acid was added dropwise at a rate of 1 mL/minute to carry out an acid treatment for 120 minutes. After the dropwise addition, the mixture was aged at 100° C. for 1 hour, and the slurry was filtered, washed with water, and dried at 105° C. for 12 hours to give a white aluminosilicate deodorant. The deodorant had a specific surface area of 250 m²/g, an acid content of 146 meq/100 g, and a composition of 0.07 Na₂O.0.93 H₂O.Al₂O₃.5.30 SiO₂.0.11 NaNO₃.4.10 H₂O.

The determination results for the ability of deodorizing ammonia are shown in Table 1, the determination results for the ability of deodorizing isovaleric acid are shown in Table 2, and the determination results for the ability of deodorizing pyridine are shown in Table 3.

### Example 2

One-hundred grams of the raw material aluminosilicate particles used in Example 1 were suspended in 900 mL of ion-exchanged water, and the resulting mixture was kept at 100° C. While stirring, 90 mL of 61% nitric acid was added dropwise at a rate of 1 mL/minute to carry out an acid treatment for 90 minutes. After the dropwise addition, the mixture was aged at 100° C. for 1 hour, and the slurry was filtered, washed with water, and dried at 105° C. for 12 hours to give a white aluminosilicate deodorant. The deodorant had a specific surface area of 110 m²/g, an acid content of 144 meq/100 g, and a composition of 0.19 Na₂O.0.81 H₂O.Al₂O₃.4.18 SiO₂.0.13 NaNO₃.3.14 H₂O.

The determination results for the ability of deodorizing ammonia are shown in Table 1, the determination results for the ability of deodorizing isovaleric acid are shown in Table 2, and the determination results for the ability of deodorizing pyridine are shown in Table 3.

### Example 3

One-hundred grams of the raw material aluminosilicate particles used in Example 1 were suspended in 900 mL of ion-exchanged water, and the resulting mixture was kept at 100° C. While stirring, 150 mL of 61% nitric acid was added dropwise at a rate of 1 mL/minute to carry out an acid treatment for 150 minutes. After the dropwise addition, the mixture was aged at 100° C. for 1 hour, and the slurry was filtered, washed with water, and dried at 105° C. for 12 hours to give a white aluminosilicate deodorant. The deodorant had a specific surface area of 442 m²/g, an acid content of 111 meq/100 g, and a composition of H₂O.Al₂O₃.20.50 SiO₂.0.22 NaNO₃.9.69 H₂O.

The determination results for the ability of deodorizing ammonia are shown in Table 1, and the determination results for the ability of deodorizing pyridine are shown in Table 3.

### Example 4

One-hundred grams of the aluminosilicate deodorant obtained in Example 1 was suspended in 900 mL of ion-exchanged water, and the resulting mixture was kept at 100° C. While stirring, an aqueous silver nitrate solution prepared by dissolving 1.55 g of silver nitrate in 30 mL of ion-exchanged water was introduced thereinto, and the mixture was aged for 1 hour. Thereafter, the aged mixture was filtered, washed with water, and dried at 105° C. for 12 hours to give a white silver-carrying aluminosilicate deodorant. The deodorant had a specific surface area of 230 m²/g, an acid content of 140 meq/100 g, and a composition of 0.02 Ag₂O.0.02 Na₂O.Al₂O₃.5.30 SiO₂.0.11 NaNO₃.5.03 H₂O.

The determination results for the ability of deodorizing ethyl mercaptan are shown in Table 4.

### Example 5

To a solution prepared by dissolving 94 g of sodium hydroxide in 1000 mL of ion-exchanged water, and further mixing 130 g of nitric acid (61%) and 124 g of a sodium aluminate solution (Na₂O=19.8% by weight, Al₂O₃=25.9% by weight, H₂O=54.3% by weight) was added 127 g of water glass (Na₂O=9.8% by weight, SiO₂=29.6% by weight, H₂O=60.6% by weight) over 1 minute, and the components were reacted at 100° C. for 8 hours. After the reaction, the formed aluminosilicate particles were filtered and washed, and dried at 105° C. for 12 hours to give a powder of raw material aluminosilicate particles. The resulting raw material aluminosilicate particles were aggregates of acicular crystals having a porous spherical form. The resulting raw material aluminosilicate particles were subjected to X-ray diffraction using an X-ray powder diffractometer [RINT2500, commercially available from Rigaku Corporation]. As a result, the aluminosilicate particles had diffraction patterns corresponding to JCPDS No. 38-513. The composition of the aluminosilicate particles was Na₂O.Al₂O₃.2.3 SiO₂.0.7 NaNO₃.1.3 H₂O, and their specific surface area was 60 m²/g.

One-hundred grams of the resulting raw material aluminosilicate particles were suspended in 900 mL of ion-exchanged water, and the resulting mixture was kept at 100° C. While stirring, 20 mL of 61% nitric acid was added dropwise at a rate of 1 mL/minute to carry out an acid treatment for 20 minutes. After the dropwise addition, the mixture was aged at 100° C. for 1 hour, and the slurry was filtered, washed with water, and dried at 105° C. for 12 hours to give a white aluminosilicate deodorant. The deodorant had a specific surface area of 115 m²/g, an acid content of 55 meq/100 g, and a composition of 0.70 Na₂O.Al₂O₃.2.05 SiO₂.0.36 NaNO₃.1.98 H₂O.

The determination results for the ability of deodorizing ammonia are shown in Table 1, and the determination results for the ability of deodorizing pyridine are shown in Table 3.

### Example 6

One-hundred grams of the aluminosilicate deodorant obtained in Example 5 was suspended in 900 mL of ion-exchanged water, and the resulting mixture was kept at 100° C. While stirring, an aqueous silver nitrate solution prepared by dissolving 1.55 g of silver nitrate in 30 mL of ion-exchanged water was introduced thereinto, and the mixture was aged for 1 hour. Thereafter, the mixture was filtered, washed with water, and dried at 105° C. for 12 hours to give a white silver-carrying aluminosilicate deodorant. The deodorant had a specific surface area of 110 m²/g, an acid content of 51 meq/100 g, and a composition of 0.01 Ag₂O.0.69 Na₂O.Al₂O₃.2.05 SiO₂.0.36 NaNO₃.2.25 H₂O.

The determination results for the ability of deodorizing ethyl mercaptan are shown in Table 4.

### Comparative Example 1

The deodorizing ability of the raw material aluminosilicate particles prepared in Example 1 was determined. The determination results for the ability of deodorizing ammonia are shown in Table 1, the determination results for the ability of deodorizing isovaleric acid are shown in Table 2, the determination results for the ability of deodorizing pyridine are shown in Table 3, and the determination results for the ability of deodorizing ethyl mercaptan are shown in Table 4.

### Comparative Example 2

The deodorizing ability of the raw material aluminosilicate particles prepared in Example 5 was determined. The determination results for the ability of deodorizing ammonia are shown in Table 1, the determination results for the ability of deodorizing isovaleric acid are shown in Table 2, the determination results for the ability of deodorizing pyridine are shown in Table 3, and the determination results for the ability of deodorizing ethyl mercaptan are shown in Table 4.

### Comparative Example 3

The deodorizing ability of a commercially available zeolite deodorant was determined. The deodorant had a composition of 0.51 Na₂O.Al₂O₃.13.8 SiO₂.0.15 H₂O. The determination results for the ability of deodorizing ammonia are shown in Table 1, the determination results for the ability of deodorizing isovaleric acid are shown in Table 2, the determination results for the ability of deodorizing pyridine are shown in Table 3, and the determination results for the ability of deodorizing ethyl mercaptan are shown in Table 4.

### Comparative Example 4

The deodorizing ability of a commercially available activated carbon was determined. The determination results for the ability of deodorizing ammonia are shown in Table 1, the determination results for the ability of deodorizing isovaleric acid are shown in Table 2, the determination results for the ability of deodorizing pyridine are shown in Table 3, and the determination results for the ability of deodorizing ethyl mercaptan are shown in Table 4.

Table 5 summarizes and collectively shows various properties of the deodorants and the like of Examples 1 to 6 and Comparative Examples 1 to 4.

TABLE 1
Odorous Component: Ammonia (Detecting
Tube: 3M for Ammonia)

| Concentration of | | | | |
|---|---|---|---|---|
| Odorous Gas | | Determination Time | | |
| (ppm) | | 0 Minute | 20 Minutes | 60 Minutes |
| Ex. | 1 | 1000 | 31 | 13 |
| | 2 | 1000 | 118 | 65 |
| | 3 | 1000 | 125 | 122 |
| | 5 | 1000 | 180 | 178 |
| Comp. Ex. | 1 | 1000 | 1000 | 1000 |
| | 2 | 1000 | 950 | 924 |
| | 3 | 1000 | 727 | 727 |
| | 4 | 1000 | 793 | 732 |

*0 Minute: Initial Concentration

TABLE 2
Odorous Component: Isovaleric Acid (Detecting
Tube: 81 for Acetic Acid)

| Concentration of | | | | |
|---|---|---|---|---|
| Odorous Gas | | Determination Time | | |
| (ppm) | | 0 Minute | 20 Minutes | 60 Minutes |
| Ex. | 1 | 60 | 6 | 4 |
| | 2 | 60 | 7 | 4 |
| Comp. Ex. | 1 | 60 | 50 | 50 |
| | 2 | 60 | 48 | 45 |
| | 3 | 60 | 20 | 12 |
| | 4 | 60 | 0 | 0 |

*0 Minute: Initial Concentration

TABLE 3
Odorous Component: Pyridine (Detecting Tube: 182 for Pyridine)

| Concentration of | | | | |
|---|---|---|---|---|
| Odorous Gas | | Determination Time | | |
| (ppm) | | 0 Minute | 20 Minutes | 60 Minutes |
| Ex. | 1 | 10 | 0 | 0 |
| | 2 | 10 | 0 | 0 |
| | 3 | 10 | 0 | 0 |
| | 5 | 10 | 0 | 0 |
| Comp. Ex. | 1 | 10 | 10 | 10 |
| | 2 | 10 | 7 | 7 |
| | 3 | 10 | 7 | 7 |
| | 4 | 10 | 1 | 1 |

*0 Minute: Initial Concentration

TABLE 4
Odorous Component: Ethyl Mercaptan (Detecting
Tube: 72 for Ethyl Mercaptan)

| Concentration of | | | | |
|---|---|---|---|---|
| Odorous Gas | | Determination Time | | |
| (ppm) | | 0 Minute | 20 Minutes | 60 Minutes |
| Ex. | 4 | 30 | 0 | 0 |
| | 6 | 30 | 0 | 0 |
| Comp. Ex. | 1 | 30 | 10 | 10 |
| | 2 | 30 | 9 | 9 |
| | 3 | 30 | 10 | 10 |
| | 4 | 30 | 0 | 0 |

*0 Minute: Initial Concentration

TABLE 5

| | Specific Surface | Acid Content | Average Particle | | Carrying | |
|---|---|---|---|---|---|---|
| | Area (m²/g) | (meq/100 g) | Size (μm) | Appearance | of Metal | State |
| Ex. 1 | 250 | 146 | 7 | White | None | Amorphous |
| Ex. 2 | 110 | 144 | 5 | White | None | Partly Crystalline |
| Ex. 3 | 442 | 111 | 7 | White | None | Amorphous |
| Ex. 4 | 230 | 140 | 7 | White | Ag | Amorphous |
| Ex. 5 | 115 | 55 | 15 | White | None | Partly Crystalline |
| Ex. 6 | 110 | 51 | 15 | White | Ag | Partly Crystalline |
| Comp. Ex. 1 | 10 | 0 | 5 | White | None | Crystalline |
| Comp. Ex. 2 | 65 | 7 | 15 | White | None | Crystalline |
| Comp. Ex. 3 | 398 | 53 | 4 | White | None | Amorphous |
| Comp. Ex. 4 | 623 | — | 51 | Black | None | Crystalline |

Comparative Examples 1 to 3 showed a lowered effect of deodorizing pyridine and ammonia each of which is an alkaline odor, and isovaleric acid and ethyl mercaptan each of which is an acidic odor. In addition, the activated carbon of Comparative Example 4 showed a lowered effect of deodorizing the alkaline odor. On the other hand, the deodorants of the present invention of Examples 1 to 4 showed a high deodorizing effect for all odorous gas components used. It can be seen from these results that the deodorant of the present invention has excellent deodorizing ability for various odorous gases.

According to the present invention, there is provided a deodorization using aluminosilicate particles, which have a wide deodorizing spectrum, are capable of effectively deodorizing an odor from various causative substances generated in daily life environment, are also safe to a human body, and furthermore exhibit excellent appearance upon application.

The present invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method of deodorization comprising allowing an odor-causing substance to coexist with an aluminosilicate particle having the composition of:
aM₂O.Al₂O₃.bSiO₂.cRₘQₙ.dH₂O,
wherein M is one or more members selected from the group consisting of Na, K and H, R is one or more members selected from the group consisting of Na, K, Ca and Mg, Q is one or more members selected from the group consisting of CO₃, SO₄, NO₃, OH and Cl, a satisfies 0<a≦1, b satisfies 1≦b≦50, c satisfies 0<c≦2, d satisfies d≧0, m satisfies 1≦m≦2, and n satisfies 1≦n≦2, and a specific surface area of 70 to 800 m²/g.

2. The method according to claim 1, wherein the acid content of the aluminosilicate particle is 20 meq/100 g or more.

3. The method according to claim 1, wherein the aluminosilicate particle is an aluminosilicate particle obtained by subjecting an aluminosilicate particle having the composition of:
wM₂O.Al₂O₃.xSiO₂.yRₘQₙ.zH₂O,
wherein M is Na, K or a mixture thereof, R is one or more members selected from the group consisting of Na, K, Ca and Mg, Q is one or more members selected from the group consisting of CO₃, SO₄, NO₃, OH and Cl, w satisfies 0.1≦w≦3, x satisfies 0.2≦x≦6, y satisfies 0<y≦2, z satisfies z≧0, m satisfies 1≦m≦2, n satisfies 1≦n≦2,
and a specific surface area of less than 70 m²/g, to an acid treatment.

4. The method according to claim 1, wherein the aluminosilicate particle carries one or more members selected from the group consisting of Ag, Cu, Zn, Fe and Ce.

5. A deodorant composition comprising the aluminosilicate particle as defined in any one of claims 1 to 4.
